(19) United States
(12) Patent Application Publication
(10) Pub. No.: US 2015/0225670 A1
(43) Pub. Date: Aug. 13, 2015

(54) RESIDUE CLEANING COMPOSITION AND METHOD

(71) Applicant: INNOVATION SERVICES, INC., Knoxville, TN (US)

(72) Inventors: Jeffrey G. HUBRIG, Knoxville, TN (US); Sam P. CROCKETT, Knoxville, TN (US)

(21) Appl. No.: 14/696,687

(22) Filed: Apr. 27, 2015

Related U.S. Application Data
(63) Continuation-in-part of application No. 14/050,704, filed on Oct. 10, 2013, now Pat. No. 9,045,718.
Application No. 14/050,704 is a continuation-in-part of application No. 13/946,625, filed on Jul. 19, 2013.
Application No. 13/946,625 is a continuation-in-part of application No. 12/869,183, filed on Aug. 26, 2010.
Application No. 12/869,183 is a continuation-in-part of application No. 11/697,933, filed on Apr. 9, 2007, now Pat. No. 7,799,234.
Application No. 11/697,933 is a continuation-in-part of application No. 11/697,921, filed on Apr. 9, 2007, now Pat. No. 7,794,606.

(51) Int. Cl. C11D 1/72; C11D 3/04; C11D 3/34

(52) U.S. Cl. CPC C11D 1/72; C11D 3/3409; C11D 3/046

(57) ABSTRACT

A medical surface cleaning and/or pre-treatment composition and a method for cleaning waste treatment system components, medical instruments surfaces, and enzyme residue-containing surfaces. The composition includes a residue cleaning agent and a substantially non-water soluble nonionic surfactant having an initial Ross-Miles foam height in an aqueous solution at 25° C. of less than 10 millimeters. A weight ratio of residue cleaning agent to surfactant on 100 wt. % active ingredient basis ranges from about 0.05:1 to about 0.5:1, and wherein the residue cleaning agent and surfactant are biodegradable.

### RELATED APPLICATIONS

This application is a continuation-in-part of application Ser. No. 14/050,704, filed Oct. 10, 2013, now allowed, which is a continuation-in-part of application Ser. No. 13/946,625, filed Jul. 19, 2013, which is a continuation-in-part of co-pending application Ser. No. 12/869,183, filed Aug. 26, 2010, which is a continuation-in-part of U.S. Pat. No. 7,799,234 issued Sep. 21, 2010 and U.S. Pat. No. 7,794,606 issued Sep. 14, 2010.

### FIELD OF THE DISCLOSURE

The present disclosure is generally directed toward compositions and methods for removing enzymatic cleaner residues and residual films from metallic and non-metallic surfaces of a medical waste treatment system or metallic and non-metallic surfaces that have come in contact with biological and/or pharmaceutical components from a medical or surgical procedure. The enzymatic cleaner residues and residual films typically result from cleaning and rinsing the metallic and non-metallic surfaces that have been in contact with the biological and/or pharmaceutical components. More specifically, the compositions and methods of the present disclosure are directed to compositions for use in hospitals and other medical facilities for removing residual traces of cleaning agents from surgical fluid medical waste treatment system surfaces, collection system surfaces, medical instruments and/or any other hard surfaces that have been initially cleaned and/or rinsed with enzymatic cleaners and other commercial cleaning compositions. The compositions are also suitable for pre-treatment of a waste medical stream to enhance the treatment of the waste stream.

### BACKGROUND AND SUMMARY

Hospitals, surgery centers and other medical treatment facilities use a variety surgical and medical instruments and devices that must be cleaned, disinfected, and reused. Such facilities have established a large number of commercially available and commonly employed cleaning agents that can leave trace amounts of the cleaning agent behind on the cleaned surface following a rinse cycle. While trace amounts of cleaning agent residuals, associated most notably with enzymatic cleaners, are within acceptable limits for washing, rinsing, disinfecting and sterilizing of surgical, medical and other devices, the residual components of such cleaning agents represent a surface contamination that may interfere with the functionality of surgical instruments and with the functionality and efficacy of components within a waste treatment system used by the same hospitals, surgery centers and other medical treatment facilities. Common practice within such facilities leads to the use of manual and automatic washer cleaning agents to clean waste treatment systems and other hard surfaces with a corresponding loss of efficacy and service life performance from critical component contamination caused by the trace amounts of cleaning agent residues.

In large medical facilities, surgical and medical instruments and devices are collected in a central location and are washed by hand and/or in an automatic washing machine before being sterilized and repackaged in a sterile container for reuse by medical personnel. Other devices that must be decontaminated may include waste collection systems and other devices that come in contact with bodily fluids and surgical waste streams.

Cleaning, not sterilization (or disinfection), is a first and most important step in any medical instrument processing protocol. Without first subjecting the instruments to a thorough, validated and standardized (and ideally automated) cleaning process, the likelihood that any disinfection or sterilization process will be effective is significantly reduced.

An automated washer/disinfector cleans and decontaminates dirty medical surgical instruments so they can be handled safely, repackaged, and sterilized for a future surgery. The danger of handling instruments contaminated with blood is obvious in this age of hepatitis, CJD and HIV. The procedures for sterilizing medical instruments are based on years of scientific testing of cleaning instruments. If surgical instruments are not clean, the procedures are ineffective. Dried blood on instruments is hazardous to the employees of the hospital and to the next surgical patient upon which the instruments are used.

Cleaning dried blood is much more difficult than cleaning dirt. Blood coagulates, which means it goes from a free-flowing liquid to a solid that contains tough, microscopic fibers called fibrin. These fibers form as the blood coagulates and jam themselves into microscopic irregularities in the surface of the stainless steel instrument. There is a physical attachment of the fibers to the surface through mechanical means, not chemical means as with traditional adhesives. The action is similar to the roots of plants growing into cracks in rocks, anchoring themselves to the surface.

Another factor that makes blood difficult to clean is its ability to become insoluble when heated. Heating causes blood to denature. Denaturing is similar to what happens to eggs cooked in a frying pan. Transparent uncooked egg whites are fairly easy to wash away, but opaque, cooked egg whites are much more difficult to remove from surfaces. Dried, uncooked egg is even more difficult to wash away, as is dried blood. the proteins in blood are similar to albumin proteins in eggs.

Current automatic washing machines are designed to use a variety of enzyme-based cleaning compositions. However, the enzyme-based cleaning compositions must be used under tightly controlled conditions in order to effectively clean and/or disinfect the medical instruments and devices. Often, the enzyme cleaning compositions leave residual enzyme components on the instruments and devices causing a need to re-clean the instruments and devices before they are reused. Also, the enzyme cleaning compositions are not always effective for cleaning hard to reach surfaces of the medical devices and instruments. If the enzyme residue is allowed to remain on the surfaces of the instruments, the enzyme residual may cause premature failure of the instruments. Accordingly, there is a need for improved cleaning compositions for use in cleaning medical instruments and enzyme residue-containing surfaces.

With regard to the foregoing needs, the disclosure provides a medical surface cleaning composition and a method for cleaning waste treatment system components, medical instrument surfaces, and enzyme residue-containing surfaces. The composition includes a residue cleaning agent and a substantially non-water soluble nonionic surfactant having an initial Ross-Miles foam height in an aqueous solution at 25° C. of less than 10 millimeters. A weight ratio of residue cleaner to surfactant on 100 wt. % active ingredient basis ranges from about 0.05:1 to about 0.5:1. The residue cleaning agent and surfactant are biodegradable.

Another embodiment of the disclosure provides a method for cleaning waste treatment system components, medical instruments surfaces, and enzyme residue-containing surfaces. The method includes applying to a surface to be cleaned a composition that includes a residue cleaning agent and a substantially non-water soluble nonionic surfactant having an initial Ross-Miles foam height in an aqueous solution at 25° C. of less than 10 millimeters. A weight ratio of residue cleaning agent to surfactant on 100 wt. % active ingredient basis ranges from about 0.05:1 to about 0.5:1. An amount of composition is sprayed onto the surface that is sufficient to effectively clean and remove residue from the surface. The surface is then rinsed with purified water to remove the composition from the cleaned surface.

An embodiment of the disclosure also includes a method for removing enzyme residue from enzyme residue-containing surfaces. The method includes applying to a surface to be cleaned a composition that includes a residue cleaning agent and a substantially non-water soluble nonionic surfactant having an initial Ross-Miles foam height in an aqueous solution at 25° C. of less than 10 millimeters. A weight ratio of residue cleaning agent to surfactant on 100 wt. % active ingredient basis ranges from about 0.05:1 to about 0.5:1. An amount of composition is sprayed onto the surface that is sufficient to effectively clean and remove residue from the surface. The surface is then rinsed with purified water to remove the composition from the cleaned surface.

Yet another embodiment of the disclosure provides a method for enhancing medical waste treatment in a medical waste treatment system. The method includes injecting into a medical waste stream, a pre-treatment composition containing (A) a residue cleaning agent selected from the group consisting of sodium lauryl sulfate, sodium lauryl ether sulfate, ammonium lauryl sulfate, ammonium lauryl ether sulfate, sophorose biosurfactant, sodium lauroyl sarcosinate, triethanolamine lauroyl-L-glutamate, sodium myristyl sarcosinate, sodium dodecyl sulfate, potassium laurate, sodium dodecane sulfonates, and sodium lauryl ethoxysulfate, (B) a substantially non-water soluble nonionic surfactant having an initial Ross-Miles foam height in an aqueous solution at 25° C. of less than 10 millimeters, and (C) a conductivity improver. A weight ratio of residue cleaning agent to surfactant on 100 wt. % active ingredient basis ranges from about 0.05:1 to about 0.5:1, wherein the amount of pre-treatment composition injected into the waste treatment system is sufficient to effectively remove residue build up on electrodes and surfaces of the medical waste treatment system and to enhance metal ion generation in the waste stream. The waste stream containing the pre-treatment composition is then flowed into the metal waste treatment system containing a metal ion generation device.

An advantage of the compositions and methods described herein is that the compositions are not highly corrosive, are low-foaming, and do not rely on the use of enzymatic agents. Enzymatic agents are highly sensitive to alkaline or acid components used in conventional cleaning compositions and to water temperatures. Another disadvantage of enzymatic cleaning agents is that such agents typically leave an enzyme cleaner residue on the cleaned surfaces that can build up over time and cause premature failure of sensitive waste treatment system components and/or medical instruments. The compositions described herein require only a single, substantially non water soluble, non-ionic surfactant and are effective for removing residual enzyme cleaner residues from the surfaces of medical instruments and other devices that were previously cleaned with enzyme cleaning agents.

Another advantage of the compositions and methods described herein is that the compositions leave substantially no detectible residue on the cleaned surfaces. A surface having no detectible residue is a surface that is visually clean to the naked eye and, over time, has no visible build up of residue upon subsequent cleaning with the same cleaning composition.

Another advantage of the compositions described here is that the compositions are optically clear and concentrates of the composition are stable over time, i.e., do not form visible precipitates in an aqueous solution of the concentrate, despite the use of a substantially non-water soluble surfactant. A further advantage of the compositions described herein is that the compositions have low or no foaming tendencies thereby enabling the compositions to effectively wet the surfaces to be cleaned without interference of foam adjacent to the surfaces. The low foaming tendency of the compositions make the compositions suitable for spray application to the surfaces under turbulent flow conditions. Other advantages may be apparent from the following detailed description.

### DETAILED DESCRIPTION OF EXEMPLARY EMBODIMENTS

For the purposes of this disclosure, the compositions described herein are made from biodegradable components. Such biodegradable components include organic compounds that are devoid of aromatic and heretocyclic groups. Accordingly, a first component of the compositions described herein is a residue cleaning agent that is provided by a biodegradable compound.

The residue cleaning agent is typically provided as a 30 wt. % solution of active ingredient. By “active ingredient” is meant the chemical compound is dissolved in a suitable solvent in order to provide the residue agent. Other solutions may be used that contain from 10 to about 50 wt. % or more of active ingredient. Accordingly, various aspects of the compositions will be discussed in terms of 100 wt. % active ingredients since the concentration of the residue cleaning agent in the cleaning composition concentrate may vary depending on the source of the residue cleaning agent.

Suitable residue cleaning agents may be selected from alkyl ether sulfates. Alkyl ether sulfates that may be used, include but are not limited to, sodium coconut alkyl sulfate, potassium coconut alkyl sulfate, potassium lauryl sulfate, sodium lauryl sulfate, sodium yellow fatty alcohol ether sulfate, tallow fatty alcohol sulfate (25 ethylene oxide), tallow fatty ether sulfate, sodium dodecyl benzene sulfonate, sodium stearyl sulfate, sodium palmityl sulfate, sodium decyl sulfate, sodium myristyl sulfate, sodium dodecyl sulfate, potassium dodecyl benzene sulfonate, potassium stearyl sulfate, potassium palmityl sulfate, potassium decyl sulfate, potassium myristyl sulfate, potassium dodecyl sulfate, and mixtures thereof.

Other examples of residue cleaning agents that may be used are sodium lauryl ether sulfate, ammonium lauryl sulfate, ammonium lauryl ether sulfate, sophorose biosurfactant, sodium lauroyl sarcosinate, triethanolamine lauroyl-L-glutamate, sodium myristyl sarcosinate, potassium laurate, sodium dodecane sulfonates, and sodium lauryl ethoxysulfate.

Without desiring to be bound by theoretical considerations, it is believed that the residue cleaning agent in the composition may react with lipid, protein, and/or enzyme residues on a surface of the medical instruments and devices to begin breaking down and denaturing both lipid and protein complexes and enzyme residues present on the surfaces of the instruments and devices. The residue cleaning agent may also interact with a bio-film layer on the surfaces of the instruments and devices through absorption and permeation to induce molecular cleavage within the bio-film structure so as to initiate adhesive failure at a boundary layer between the bio-film structure and the underlying substrate surface. Once adhesion failure is induced by the residue cleaning agent, the organic material on the surfaces of the instruments and devices may be readily rinsed from the instruments and devices with plain water.

A particularly useful residue cleaning agent for use in the compositions described herein is sodium lauryl sulfate (SLS). SLS is often referred to as an anionic surfactant. However, in the compositions described herein, SLS has more of a detergent effect. The compositions described herein may contain an amount of SLS, on an active ingredient basis, that is effective to promote permeation, solubilization and mobilization of protein, lipid structures, and/or enzyme residues, thereby releasing the bio-film and enzyme residues from surface of the devices and instruments. Accordingly, the amount of residue cleaning agent in the washing solutions described herein may range from 5 mL per liter of total washing liquid to about 150 mL per liter of total washing liquid based on a 30 wt. % active solution of residue cleaning agent.

The second important component of the compositions described herein is a nonionic, non-water soluble surfactant having an initial Ross-Miles foam height in an aqueous solution at 25° C. of less than 10 millimeters, such as less than about 7 millimeters, or less than about 5 millimeters, and desirably less than about 3 millimeters. The Ross-Miles foam height of a compound is determined according to ASTM D1173 using a 0.1 wt. % aqueous solution of the compound at a temperature of 25° C.

The nonionic surfactants which may be used may be selected from biodegradable, linear and branched alkoxylated alcohols. Still further illustrative examples of nonionic surfactants include primary and secondary linear and branched alcohol ethoxylates, such as those based on C₆ to C₁₈ alcohols which further include an average of from 1 to 80 moles of ethoxylation per mol of alcohol.

Further examples of useful nonionic surfactants include secondary C₁₂ to C₁₈ alcohol ethoxylates, including those which have from about 3 to about 10 moles of ethoxylation. Further exemplary nonionic surfactants include linear primary C₁₁ to C₁₅ alcohol ethoxylates, including those which have from about 3 to about 10 moles of ethoxylation. Other surfactants include linear C₁₁ alcohol with 1 mole (average) of ethylene oxide. Examples include polyoxyethylene (2) cetylether and polyoxyetylene (2) oleylether.

Other examples of useful nonionic surfactants include polyethylene-block poly(ethylene glycol) surfactants having an number average molecular weight of about 875; and poly(ethylene glycol)-block poly(propylene glycol)-block-polyethylene glycol) copolymers having number average molecular weights ranging from about 1100 to about 3500.

Still other non-ionic surfactants which may be used include: fatty acid monoalkylolamide ethoxylates, fatty amine alkoxylates and fatty acid glyceryl ester ethoxylates. Other non-ionic compounds suitable for inclusion in compositions of the disclosed embodiments include mixed ethylene oxide propylene oxide block copolymers, low relative molecular mass polyethylene glycols, ethylene glycol monoesters, amine oxides and alkyl polyglycosides, alkyl sugar esters including alkyl sucrose esters and alkyl oligosaccharide ester, alkyl capped polyvinyl alcohol and alkyl capped polyvinyl pyrrolidone.

Of the foregoing nonionic surfactants, one or more ethoxylated linear or branched alcohol nonionic surfactants having an initial Ross-Miles foam height of a 0.1 wt % aqueous solution at 25° C. ranging from about 1 to less than about 10 millimeters, such as from 2 to less than about 7 millimeters, and particularly from about 2 to less than about 5 millimeters may provide the most suitable foam inhibiting effects in combination with the residue cleaning agent. Accordingly, the surfactant may be a single surfactant with an initial Ross-Miles foam height of a 0.1 wt % aqueous solution at 25° C. of less than 10 millimeters, or a combination of surfactants having the same initial Ross-Miles foam height. The amount of nonionic surfactant relative to the amount of residue cleaning agent on a weight ratio basis (100 wt. % active ingredient) in the compositions described herein may range from about 2:1 to about 20:1. For example, cleaning composition concentrates may include a weight ratio of surfactant to residue cleaning agent of from about 3:1 to about 8:1 or from about 4:1 to about 6:1. For the purposes of this disclosure, all references to the nonionic surfactant is with respect to a surfactant that is 100 wt. % active ingredient.

An optional component of the compositions described herein is an aqueous solvent, such as water. Washing solution concentrates as described herein may typically contain a major amount of water. Accordingly, the compositions may contain from about 50 to about 99.9 volume percent water. For example, the compositions from about 60 to about 95 volume percent water. Other compositions may include from about 75 to about 90 volume percent water. Solubilizing agents may be included in the compositions to aid in solubilizing the components of the composition. For example, concentrates containing the surfactants and residue cleaning agent may require dispersing or solubilizing agents to provide uniform solution concentrates that may be diluted upon use to provide the pretreatment and conditioning. Such solubilizing or dispersing agents may include, but are not limited to, alcohols, glycols, glycerines, and the like. The amount of solubilizing or dispersing agent in the compositions described herein may range from about 2 to about 10 percent by volume based on the total volume of the concentrate.

Pure water typically has a conductivity well below 1 μ-Siemens/cm. However, medical waste materials such as blood and surgical fluids may increase the conductivity of a medical waste stream containing such materials. However, if the conductivity is below about 6 milli-Siemens/cm, the generation of ions in an ion infusion system for treating the waste stream as disclosed in U.S. Pat. Nos. 7,794,606 and 7,799,234 and in U.S. Patent Publication Nos. 2011/0290740 and 2013/0298946, the disclosures of which are incorporated herein by reference.

Accordingly, a conductivity improver may be included in the composition used to pre-treat the medical waste stream in order to enhance production of metal ions in the waste stream. A wide variety of conductivity improvers may be used, including, but not limited to, alkali and alkaline earth metal salts, ammonium salts and the like. pH adjustment agents such as sodium hydroxide or potassium hydroxide may be used to increase the conductivity, however, the use of such compounds may be limited in order to maintain a pH of the waste stream at about 8.0 to about 9.5. Thus conductivity improvers that may be used include ammonium sulfate, calcium chloride, sodium chloride, lithium chloride, magnesium chloride, magnesium sulfate, potassium carbonate, potassium chloride, potassium hydrogen carbonate, potassium hydrogen phosphate, potassium hydroxide, sodium hydroxide, potassium iodide, potassium nitrate, potassium sulfate, sodium bromide, sodium carbonate, and the like. A particularly useful conductivity improver is sodium chloride.

The amount of conductivity improver in the pre-treatment composition may range from about 5 to about 20 percent by weight based on a total weight of the active ingredients of pre-treatment composition. The pre-treatment composition includes the residue cleaning agent, the nonionic, non-water soluble surfactant, the conductivity improver and water as described above. Accordingly, the pre-treatment composition may have a conductivity ranging from about 60 to about 120 milli-Siemens/cm. The amount of pre-treatment solution injected into a medical waste stream in order to enhance the conductivity of the waste stream is an amount sufficient to sufficient to effectively remove residue build up on electrodes and surfaces of the medical waste treatment system and to enhance metal ion generation in the waste stream. Thus, if the conductivity of the waste stream, prior to injecting the pre-treatment solution, is below about 6 milli-Siemens/cm, the pre-treatment composition may be sufficient to increase the conductivity of the waste stream to from about 6 to about 15 milli-Siemens/cm or higher.

Other components which may be present in the compositions described herein may include but are not limited to pH adjustment agents, biocides, bacteriacides, sterilization agents, antifungal agents, germicides, dyes, chelating agents, and the like.

The major components of the compositions described herein may promote a pH that is slightly acidic to neutral. However, the compositions may be more effective for the automatic washing machines used in hospitals if the compositions are slightly alkaline. According, a pH adjustment agent may be added to the composition to provide a pH in the range of from about 6.5 to about 10.0. A more desirable pH of the compositions described herein may range from about 8.5 to about 9.5.

A suitable pH adjustment agent may be selected from weak bases such as, ammonium hydroxide, 2-aminopropanoic acid, ammonia, magnesium hydroxide, methylamine, ethylamine, dimethylamine, trimethylamine, pyridine, glycine, hydrazine, and the like. Accordingly, compositions as describe herein may include from about 0.01 to about 1.0 percent by weight of the pH adjustment agent based on a total weight of the composition. Washing solution concentrates may contain from about 0.01 to about 0.5 weight percent of the pH adjustment agent.

The compositions described herein may be particularly suitable for use in an automatic washing and/or disinfection machine used in hospitals to clean medical instruments that have been previously hand washed with other cleaning agents. The low or no foaming tendencies of the compositions make them particularly suitable for such spray washing applications. Other uses of the compositions described herein may include cleaning other surfaces and devices that have been initially cleaned with enzyme cleaning agents.

Once the surfaces of the instruments and devices are clean, the composition described herein may be readily rinsed from the surfaces of the devices so as to leave substantially no visually detectible composition residue or organic material on the surfaces.

An advantage of the compositions described herein is the compositions do not require the addition of antifoam agents. A residual cleaning agent such as SLS tends to foam excessively under turbulent conditions in an aqueous stream. However, use of a sufficient amount of surfactant having an initial Ross-Miles foam height in an aqueous solution at 25° C. of less than 10 millimeters provides sufficient foam inhibition in a turbulent aqueous stream. Accordingly, the combination of residual cleaning agent and surfactant may be used in a flowing stream under extremely turbulent conditions, with or without spray nozzles without excessive foam generation enabling the composition to be turbulently sprayed into an automatic washing machine or used with other high pressure washing systems.

With regard to compositions containing the residual cleaning agent (RCA) and the surfactant component described above, the ranges listed in Table 1 may be used in automatic washing machines for medical facilities. Higher ratios of RCA to surfactant (Compositions 1-4) may be used where the generation of foam are minimal By contrast, Compositions 6-10 may be used where turbulence and foaming are problematic with regard to adequate cleaning. For example, Composition 1 having a weight ratio of RCA to surfactant of about 2.7:1 on a 100 wt. % active basis may be injected into an automatic washing machine that is used without first hand washing or rinsing of the medical instruments. Composition 10, having a weight ratio of RCA to surfactant of about 0.016:1 on a 100 wt. % active basis, may be used in automatic washing machines to clean surfaces containing dried blood or other medical waste materials such as ocular fluids and the like subsequent to hand washing the medical instruments. Selection of compositions between Compositions 1 and 10 may be made for particular applications depending on washing conditions, size of the automatic washing machines and other factors of machine design that may cause foaming in the washing machine.

TABLE 1

| | Residual Cleaning | | RCA |
|---|---|---|---|
| | Agent | | (100 wt. % active)/ |
| Compo- | Solution (RCA), | Surfactant, | Surfactant |
| sition | 30 wt. % active | 100 wt. % active | (100 wt. % active) |
| 1 | 90 | 10 | 2.7 |
| 2 | 80 | 20 | 1.2 |
| 3 | 70 | 30 | 0.7 |
| 4 | 60 | 40 | 0.45 |
| 5 | 50 | 50 | 0.3 |
| 6 | 40 | 60 | 0.2 |
| 7 | 30 | 70 | 0.128 |
| 8 | 20 | 80 | 0.075 |
| 9 | 10 | 90 | 0.033 |
| 10 | 5 | 95 | 0.016 |

Compositions 1-10 may be diluted in water or a saline solution before use of the compositions in an automatic washing machine. In the table, all weights are in grams of ingredients.

TABLE 2
Composition Formulations Component gram weight additions for each total Solution Concentration Level Percentage

| | 0.25% | | 0.50% | | 1.00% | | 2.00% | |
|---|---|---|---|---|---|---|---|---|
| | SLS, 30 | | SLS, 30 | | SLS, 30 | | SLS, 30 | |
| | wt. % | Non-Ionic | wt. % | Non-Ionic | wt. % | Non-Ionic | wt. % | Non-Ionic |
| Comp. | active | Surfactant | active | Surfactant | active | Surfactant | active | Surfactant |
| 1 | 1.0215 | 0.1135 | 2.0430 | 0.2270 | 4.0860 | 0.4540 | 8.1720 | 0.9080 |
| 2 | 0.9080 | 0.2270 | 1.8160 | 0.4540 | 3.6320 | 0.9080 | 7.2640 | 1.8160 |
| 3 | 0.7945 | 0.3405 | 1.5890 | 0.6810 | 3.1780 | 1.3620 | 6.3560 | 2.7240 |
| 4 | 0.6810 | 0.4540 | 1.3620 | 0.9080 | 2.7240 | 1.8160 | 5.4480 | 3.6320 |
| 5 | 0.5675 | 0.5675 | 1.1350 | 1.1350 | 2.2700 | 2.2700 | 4.5400 | 4.5400 |
| 6 | 0.4540 | 0.6810 | 0.9080 | 1.3620 | 1.8160 | 2.7240 | 3.6320 | 5.4480 |
| 7 | 0.3405 | 0.7945 | 0.6810 | 1.5890 | 1.3620 | 3.1780 | 2.7240 | 6.3560 |
| 8 | 0.2270 | 0.9080 | 0.4540 | 1.8160 | 0.9080 | 3.6320 | 1.8160 | 7.2640 |
| 9 | 0.1135 | 1.0215 | 0.2270 | 2.0430 | 0.4540 | 4.0860 | 0.9080 | 8.1720 |
| 10 | 0.0568 | 1.0783 | 0.1135 | 2.1565 | 0.2270 | 4.3130 | 0.4540 | 8.6260 |
| | 4.00% | | 6.00% | | 8.00% | | 10.00% | |
| | SLS, 30 | | SLS, 30 | | SLS, 30 | | SLS, 30 | |
| | wt. % | Non-Ionic | wt. % | Non-Ionic | wt. % | Non-Ionic | wt. % | Non-Ionic |
| Comp. | active | Surfactant | active | Surfactant | active | Surfactant | active | Surfactant |
| 1 | 16.3440 | 1.8160 | 24.5160 | 2.7240 | 32.6880 | 3.6320 | 40.8600 | 4.5400 |
| 2 | 14.5280 | 3.6320 | 21.7920 | 5.4480 | 29.0560 | 7.2640 | 36.3200 | 9.0800 |
| 3 | 12.7120 | 5.4480 | 19.0680 | 8.1720 | 25.4240 | 10.8960 | 31.7800 | 13.6200 |
| 4 | 10.8960 | 7.2640 | 16.3440 | 10.8960 | 21.7920 | 14.5280 | 27.2400 | 18.1600 |
| 5 | 9.0800 | 9.0800 | 13.6200 | 13.6200 | 18.1600 | 18.1600 | 22.7000 | 22.7000 |
| 6 | 7.2640 | 10.8960 | 10.8960 | 16.3440 | 14.5280 | 21.7920 | 18.1600 | 27.2400 |
| 7 | 5.4480 | 12.7120 | 8.1720 | 19.0680 | 10.8960 | 25.4240 | 13.6200 | 31.7800 |
| 8 | 3.6320 | 14.5280 | 5.4480 | 21.7920 | 7.2640 | 29.0560 | 9.0800 | 36.3200 |
| 9 | 1.8160 | 16.3440 | 2.7240 | 24.5160 | 3.6320 | 32.6880 | 4.5400 | 40.8600 |
| 10 | 0.9080 | 17.2520 | 1.3620 | 25.8780 | 1.8160 | 34.5040 | 2.2700 | 43.1300 |

With regard to Table 2, generally useful compositions for a wide variety of applications may fall within Compositions 3-5 over a range of dilution of 0.25 to 10% by weight. Other useful compositions may fall within Compositions 8-10 over a range of dilution of 0.25 to 10% by weight. The actual weight percent of active ingredient on 100 wt. % basis for each of the formulations shown in Table 2 may be determined by multiplying the amount of SLS by 0.30, adding the amount of surfactant and dividing the sum by the total weight of SLS, surfactant, and diluent.

For pre-treatment solutions for use in medical waste treatment systems as described above, a typical composition may include from about 3 to about 5 wt. % residue cleaning agent (30 wt. % active solution), from about 3 to about 5 wt. % non-ionic surfactant, from about 5 to about 20 wt. % conductivity improver, trace amounts of colorants, fragrances, antifreeze compounds, and the like and the balance, purified water.

It is contemplated, and will be apparent to those skilled in the art from the preceding description that modifications and/or changes may be made in the embodiments of the disclosure. Accordingly, it is expressly intended that the foregoing description is illustrative of exemplary embodiments only, not limiting thereto, and that the true spirit and scope of the present disclosure be determined by reference to the appended claims.

1. A medical surface cleaning composition devoid of an antifoam agent and enzymatic cleaning agent comprising (A) a residue cleaning agent selected from the group consisting of sodium lauryl sulfate, sodium lauryl ether sulfate, ammonium lauryl sulfate, ammonium lauryl ether sulfate, sophorose biosurfactant, sodium lauroyl sarcosinate, triethanolamine lauroyl-L-glutamate, sodium myristyl sarcosinate, sodium dodecyl sulfate, potassium laurate, sodium dodecane sulfonates, and sodium lauryl ethoxysulfate and (B) a substantially non-water soluble nonionic surfactant having an initial Ross-Miles foam height in an aqueous solution at 25° C. of less than 10 millimeters, wherein a weight ratio of residue cleaner to surfactant on 100 wt. % active ingredient basis ranges from about 0.05:1 to about 0.5:1, and wherein the residue cleaning agent and surfactant are biodegradable.

2. The composition of claim 1, further comprising water.

3. The composition of claim 1, wherein the weight ratio of residue cleaning agent to surfactant in the composition on 100 wt. % active ingredient basis ranges from about 0.075:1 to about 0.3:1.

4. The composition of claim 1, wherein the surfactant comprises a polyether polyol non-ionic surfactant having an initial Ross-Miles foam height in an aqueous solution at 25° C. of less than 5 millimeters.

5. The composition of claim 1, wherein the residue cleaning agent comprises sodium lauryl sulfate.

6. The composition of claim 1, further comprising from about 5 to about 20 percent by weight based on a total weight of the composition of a conductivity improver.

7. The composition of claim 6, wherein the conductivity improver is selected from the group consisting of alkali and alkaline earth metal salts.

8. The composition of claim 7, wherein the composition has a conductivity ranging from about 60 to about 210 milli-Siemens/cm.

9. A method for cleaning medical waste treatment system components to remove residue and film formation on surfaces of the medical waste treatment system, comprising:
injecting into the waste treatment system a composition devoid of an antifoam agent and enzymatic cleaning agent comprising (A) a residue cleaning agent selected from the group consisting of sodium lauryl sulfate, sodium lauryl ether sulfate, ammonium lauryl sulfate, ammonium lauryl ether sulfate, sophorose biosurfactant, sodium lauroyl sarcosinate, triethanolamine lauroyl-L-glutamate, sodium myristyl sarcosinate, sodium dodecyl sulfate, potassium laurate, sodium dodecane sulfonates, and sodium lauryl ethoxysulfate, and (B) a substantially non-water soluble nonionic surfactant having an initial Ross-Miles foam height in an aqueous solution at 25° C. of less than 10 millimeters, wherein a weight ratio of residue cleaning agent to surfactant on 100 wt. % active ingredient basis ranges from about 0.05:1 to about 0.5:1, wherein the amount of composition injected into the waste treatment system is sufficient to effectively clean and remove residue from surfaces of the medical waste treatment system; and
rinsing the surfaces with purified water to remove the composition from the cleaned surfaces.

10. The method of claim 9, wherein the residue cleaning agent comprises sodium lauryl sulfate.

11. A method for enhancing medical waste treatment in a medical waste treatment system, comprising:
injecting into a medical waste stream, a pre-treatment composition comprising (A) a residue cleaning agent selected from the group consisting of sodium lauryl sulfate, sodium lauryl ether sulfate, ammonium lauryl sulfate, ammonium lauryl ether sulfate, sophorose biosurfactant, sodium lauroyl sarcosinate, triethanolamine lauroyl-L-glutamate, sodium myristyl sarcosinate, sodium dodecyl sulfate, potassium laurate, sodium dodecane sulfonates, and sodium lauryl ethoxysulfate, (B) a substantially non-water soluble nonionic surfactant having an initial Ross-Miles foam height in an aqueous solution at 25° C. of less than 10 millimeters, and (C) a conductivity improver, wherein a weight ratio of residue cleaning agent to surfactant on 100 wt. % active ingredient basis ranges from about 0.05:1 to about 0.5:1, wherein the amount of pre-treatment composition injected into the waste treatment system is sufficient to effectively remove residue build up on electrodes and surfaces of the medical waste treatment system and to enhance metal ion generation in the waste stream; and
flowing the waste stream containing the pre-treatment composition into the metal waste treatment system containing a metal ion generation device.

12. The method of claim 11, wherein the conductivity improver is present in an amount ranging from about 5 to about 20 percent by weight of the total weight of the pre-treatment composition

13. The method of claim 11, wherein the conductivity improver is selected from the group consisting of alkali and alkaline earth metal salts.

14. The method of claim 13, wherein the pre-treatment composition has a conductivity ranging from about 60 to about 210 milli-Siemens/cm.

15. The method of claim 13, wherein the conductivity improver comprises sodium chloride.

16. The method of claim 11, wherein the pre-treatment composition is provided in an amount effective to increase the conductivity of a medical waste stream from about 6 to about 15 milli-Siemens/cm or more.
